(19) United States
(12) Patent Application Publication
(10) Pub. No.: US 2006/0264380 A1
(43) Pub. Date: Nov. 23, 2006

(54) Compounds and Methods for Promoting Angiogenesis

(76) Inventors: Mats Hellstrom, Goteborg (SE); Linda Karlsson, Goteborg (SE); Elisabeth Wallgard, Goteborg (SE)

Correspondence Address: ALBIHNS STOCKHOLM AB, BOX 5581, LINNEGATAN 2, SE-114 85 STOCKHOLM; SWEDENn, STOCKHOLM, SE

(21) Appl. No.: 11/306,973

(22) Filed: Jan. 18, 2006

Related U.S. Application Data
(63) Continuation-in-part of application No. PCT/SE04/01146, filed on Jul. 21, 2004.
(60) Provisional application No. 60/488,345, filed on Jul. 21, 2003.
(60) Provisional application No. 60/593,548, filed on Jan. 25, 2005.

(30) Foreign Application Priority Data
Jul. 21, 2003 (SE) 0302111-0

(51) Int. Cl. A61K 38/04; A61K 31/18

(52) U.S. Cl. 514/19; 514/602

(57) ABSTRACT

Angiogenesis may be initiated or increased through the use of gamma-secretase inhibitors. The gamma-secretase inhibitor can be a dipeptide class, sulfonamide class, transition state mimic class, benzodiazepine class, or benzocaprolactam class gamma secretase inhibitor. Methods for initiating and increasing angiogenesis are used for disease prevention and treatment as well as for generating research models.

### CROSS REFERENCE TO RELATED APPLICATIONS

This application is a continuation-in-part of International Application PCT/SE2004/001146, filed Jul. 21, 2004, designating the United States of America, which claims the benefit of Provisional Application No. 60/488,345, filed Jul. 21, 2003, and Swedish Patent Application No. 0302111-0, filed Jul. 21, 2003, and also claims the benefit of U.S. Patent Application No. 60/593,548, filed Jan. 25, 2005.

### FIELD OF THE INVENTION

The invention relates to the field of angiogenesis. In particular, the invention provides compounds and methods useful in the treatment of diseases or conditions related to angiogenic abnormalities.

### BACKGROUND OF THE INVENTION

Angiogenesis is a fundamental process required for the normal growth and development of tissues, and involves the proliferation of new capillaries from preexisting blood vessels. Under normal physiological conditions, humans or animals only undergo angiogenesis in very specific situations and angiogenesis is tightly controlled through a highly regulated system of angiogenic stimulators and inhibitors. Deviation from such a tight control often leads to or is associated with disease.

Angiogenesis is a prerequisite for the development and differentiation of the vascular tree, as well as for a wide variety of fundamental physiological processes including embryogenesis, somatic growth, tissue and organ repair and regeneration, cyclical growth of the corpus luteum and endometrium, and development and differentiation of the nervous system. In the female reproductive system, angiogenesis occurs in the follicle during its development, in the corpus luteum following ovulation and in the placenta to establish and maintain pregnancy. Angiogenesis additionally occurs as part of the body's repair processes, e.g. in the healing of wounds and fractures. Angiogenesis is also a factor in tumor growth, since a tumor must continuously stimulate growth of new capillary blood vessels in order to grow.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

While persistent, unregulated angiogenesis occurs in numerous disease states, insufficient or nonexistent angiogenesis can also be a serious medical problem. Promoting angiogenesis is desirable in situations where vascularization is to be established or extended, for example after tissue or organ transplantation, or to stimulate establishment of collateral circulation in tissue infarction or arterial stenosis, such as in coronary heart disease and thromboangitis obliterans. Enhancing angiogenic activity may also be useful in treating ischemic conditions, including cardiovascular and limb ischemia. Finally, materials or methods that initiate or increase angiogenesis could potentially also be used to create research models with greater-than-normal angiogenesis.

The protease gamma-secretase is a complex of at least four proteins: presenilin 1 (PS 1), nicastrin, APH-1, and PEN-2. Gamma-secretase has more than one enzymatic activity cleaving multiple substrates. It is also involved in processing the Notch receptor, part of a signalling pathway critical for embryonic development. The importance of this pathway is seen in knockout PS-1 mice which die in utero or shortly after birth, understood to be at least partially due to PS-1's role in normal or sufficient angiogenesis. Researchers have thus found it desirable to both further define gamma-secretase itself, as well as to identify compounds which interact with gamma-secretase, such as inhibitors.

Gamma-secretase or gamma-secretase pathway inhibitors are grouped into five classes of compounds. See, for example, Michael S. Wolfe, Therapeutic Strategies for Alzheimer's Disease, Drug Discovery, Vol 1, pp 859-866 (November 2002). The first class comprises small, organic molecules that resemble an intermediate of enzyme catalysis such as compounds shown in Formulas I VI (see FIGS. 28a and b). These class-one compounds are referred to as transition-state mimics.

The second group consists of benzodiazepines such as the compound represented by Formula VII.

The third group consists of sulphonamides and sulfones such as the compounds of Formulas VIII and IX.

The fourth group consists of dipeptides and semi-peptidic inhibitors such as the compounds shown in Formulas X and XI. The compound of Formula X is (N-[N-(3,5-Difluorophenacetyl-L-alanyl)]-S-phenylglycine t-Butyl Ester or DAPT, a cell-permeable dipeptide protease inhibitor, one of the known gamma-secretase inhibitors which blocks Notch signaling (Micchelli, C. A. et al., 2003, gamma-secretase/presenilin inhibitors for Alzheimer's disease phenocopy Notch mutations in Drosophila, FASEB J. 17, 79-81). Difluorophenacetyl-L-alanyl)]-S-phenylglycine The fifth group consists of benzocaprolactams, for example, the compound of Formula XII.

In addition to its interest as a research tool, numerous diseases can be linked to gamma-secretase or the gamma-secretase pathway. For example, gamma-secretase is involved in Alzheimer's Disease (“Alzheimer's”). Alzheimer's is characterized by the formation of plaques. The processing of precursor proteins which can result in the plaques is known to involve gamma-secretase.

Because of their utility in the treatment and prevention of Alzheimer's alone, gamma-secretase and gamma-secretase pathway inhibitors have been identified and developed. But while significant research has been done identifying gamma-secretase, its pathway, and inhibitors thereof, as well as diseases and abnormal conditions related to gamma-secretase, there remains a need in the art to completely identify the mechanisms of action of gamma-secretase and utilize this knowledge to improve modern medicine.

### SUMMARY OF THE INVENTION

It is therefore an object of the present invention to provide materials or methods that initiate or increase angiogenesis. It is further an objection of the present invention to describe new uses for gamma-secretase inhibitors. According to the invention, gamma-secretase inhibitors have surprisingly been shown to increase angiogenesis. This novel elucidation of activity is exploited in treatments for angiogenesis and related conditions.

According to a first embodiment of the invention, a compound is provided which comprises a pharmaceutically-effective amount of a gamma-secretase pathway inhibitor and which initiates or increases angiogenesis. The gamma-secretase pathway inhibitor can comprises a dipeptide class gamma-secretase pathway inhibitor such as DAPT, a sulfonamide class gamma-secretase inhibitor such as 1-(S)-endo-N-(1,3,3)-Trimethylbicyclo[2.2.1]hept-2-yl)-4-fluorophenyl Sulfonamide, a transition state mimic class gamma-secretase inhibitor such as WPE-III31C, a benzodiazepine class gamma-secretase pathway inhibitor such as S-3-[N′-(3,5-difluorophenyl-alpha-hydroxyacetyl)-L-alanilyl]amino-2,3-dihydro-1-methyl-5-phenyl-1H-1,4-benzodiazepin-2-one, or a benzocaprolactam class gamma-secretase inhibitor such as (N)-[(S)-2-hydroxy-3-methyl-butyryl]-1-(L-alaninyl)-(S)-1-amino-3-methyl-4,5,6,7-tetrahydro-2H-3-benzazepin-2-one. The compound could be formulated in a pharmaceutical composition, optionally including a pharmaceutically acceptable carrier or adjuvant.

According to a further embodiment of the invention, a method of influencing a disease state in a cell, a group of cells, or an organism is provided, which comprises administering at least one of a gamma-secretase inhibitor or a gamma-secretase pathway inhibitor to the cell, group of cells, or organism, wherein the disease is selected from the group consisting of atherosclerosis, hemangioma, hemangioendothelioma, vascular malformations, warts, pyogenic granulomas, hair growth, Kaposi's sarcoma, scar keloids, allergic edema, neoplasms, psoriasis, decubitus or stasis ulcers, gastrointestinal ulcers, dysfunctional uterine bleeding, follicular cysts, ovarian hyperstimulation, endometriosis, neoplasms, preeclampsia, placental insufficiency, respiratory distress, ascites, peritoneal sclerosis, adhesion formation, metastatic spreading, coronary artery disease, ischemic heart disease, ischemic limb disease, obesity, rheumatoid arthritis, synovitis, bone destruction, cartilage destruction, osteomyelitis, pannus growth, osterphyte formation, cancer, aseptic necrosis, impaired fracture healing, hepatitis, pneumonia, glomerulonephritis, asthma, nasal polyps, liver regeneration, pulmonary hypertension, systemic hypertension, diabetes, retinopathy of prematurity, diabetic retinopathy, choroidal disorders, intraocular disorders (e.g. age related macular degeneration), leukomafacia, stroke, vascular dementia, disease, thyroiditis, thyroid enlargement, thyroid pseudocyst, tumor metastasis, lymphoproliferative disorders, lympgoedema, AIDS, and hematologic malignancies.

According to a further embodiment of the invention, a method of increasing the angiogenic process in a cell, a group of cells, or an organism is provided which comprises administering a pharmaceutical composition which comprises a pharmaceutically effective amount of at least one gamma-secretase inhibitor or gamma-secretase pathway inhibitor to the cell, group of cells, or organism. The pharmaceutical composition may be administered to prevent, treat, or cure a condition selected from the group consisting of atherosclerosis, hemangioma, hemangioendothelioma, vascular malformations, warts, pyogenic granulomas, hair growth, Kaposi's sarcoma, scar keloids, allergic edema, neoplasms, psoriasis, decubitus or stasis ulcers, gastrointestinal ulcers, dysfunctional uterine bleeding, follicular cysts, ovarian hyperstimulation, endometriosis, neoplasms, preeclampsia, placental insufficiency, respiratory distress, ascites, peritoneal sclerosis, adhesion formation, metastatic spreading, coronary artery disease, ischemic heart disease, eschemic limb disease, obesity, rheumatoid arthritis, synovitis, bone destruction, cartilage destruction, osteomyelitis, pannus growth, osterphyte formation, cancer, aseptic necrosis, impaired fracture healing, hepatitis, pneumonia, glomerulonephritis, asthma, nasal polyps, liver regeneration, pulmonary hypertension, systemic hypertension, diabetes, retinopathy of prematurity, diabetic retinopathy, choroidal disorders, intraocular disorders (e.g. age related macular degeneration), leukomafacia, stroke, vascular dementia, disease, thyroiditis, thyroid enlargement, thyroid pseudocyst, tumor metastasis, lymphoproliferative disorders, lympgoedema, AIDS, and hematologic malignancies.

According to a further embodiment of the invention, a method for initiating or increasing angiogenesis in a cell, a group of cells, a tissue, or an organism is provided, which comprises inhibiting a gamma-secretase pathway in said cell, group of cells, tissue or organism.

According to a further embodiment of the invention, a method for initiating or increasing angiogenesis in a cell, a group of cells, a tissue, or an organism is provided, which comprises inhibiting gamma-secretase in said cell, group of cells, tissue or organism. This may be effected by administering an antibody against gamma-secretase to said cell, group of cells, tissue or organism, or optionally by delivering a vector to an organism, wherein said vector comprises a polynucleotide encoding at least one gamma-secretase inhibitor, operatively linked to a suitable promoter. In such a case the promoter may be a tissue- or organ-specific promoter specific for a tissue or organ in which angiogenesis is to be initiated or increased.

According to a further embodiment of the invention, a method for screening for a substance which initiates or increases angiogenesis is provided, which comprises measuring an activity of a gamma-secretase pathway in the presence of a candidate compound in a suitable model, measuring an activity of a gamma-secretase pathway in the absence of a candidate compound, and comparing the activity in the presence of a candidate compound with the activity in the absence of the candidate compound, wherein a change in activity indicates that the candidate initiates or increases angiogenesis.

According to a further embodiment of the invention, a medicament which initiates or increases angiogenesis is provided, which comprises a pharmaceutically-effective amount of a gamma-secretase pathway inhibitor and a pharmaceutically-effective amount of at least one pro-angiogenic therapy. The gamma-secretase pathway inhibitor and the pro-angiogenic therapy may be provided together as a single pharmaceutical composition. The gamma-secretase pathway inhibitor may comprise an inhibitor selected from the group consisting of a dipeptide class gamma-secretase pathway inhibitor, a sulfonamide class gamma-secretase inhibitor, a transition state mimic class gamma-secretase inhibitor, a benzodiazepine class gamma-secretase inhibitor, and a benzocaprolactam class gamma-secretase inhibitor. For example, the gamma-secretase pathway inhibitor may be selected from the group consisting of DAPT, 1-(S)-endo-N-(1,3,3)-Trimethylbicyclo[2.2.1]hept-2-yl)-4-fluorophenyl Sulfonamide, WPE-III31C, S-3-[N′-(3,5-difluorophenyl-alpha-hydroxyacetyl)-L-alanilyl]amino-2,3-dihydro-1-methyl-5-phenyl-1H-1,4-benzodiazepin-2-one, and (N)-[(S)-2-hydroxy-3-methyl-butyryl]-1-(L-alainyl)-(S)-1-amino-3-methyl-4,5,6,7-tetrahydro-2H-3-benzazepin-2-one. The pro-angiogenic therapy may be selected from the group consisting of VEGF-A, VEGF-B, VEGF-C, VEGF-D, FGF-1, FGF-2, FGF-4, HiFalpha, and HGF.

As used herein “gamma-secretase inhibitor” means any material or compound that, e.g., binds to, partially or totally blocks activity, decreases, prevents, delays activation, inactivates, desensitizes, or down regulates the activity or expression of gamma-secretase or the gamma-secretase pathway. Inhibitors include genetically modified versions of gamma-secretase proteins, e.g., versions with altered activity, as well as naturally occurring and synthetic ligands, antagonists, agonists, antibodies, small chemical molecules and the like. Inhibitor, as the term is used herein, includes but is not limited to an antagonist.

The present invention encompasses compounds and compositions which have are pharmaceuticals or have a pharmaceutical effect. The compounds of the invention may be admixed, encapsulated, conjugated or otherwise associated with other molecules, molecule structures or mixtures of compounds for assisting in uptake, distribution and/or absorption. They encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The compositions of the present invention may additionally contain other adjunct components conventionally found in pharmaceutical compositions, at their art-established usage levels. Thus, for example, the compositions may contain additional, compatible, pharmaceutically-active materials such as, for example, local anesthetics, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the active compound.

The phrase “pharmaceutically acceptable” refers to molecular entities and compositions that are physiologically tolerable and do not typically produce an allergic or similar untoward reaction, such as gastric upset, dizziness and the like, when administered to a patient.

The phrase “pharmaceutically effective amount” is used herein to mean an amount sufficient to initiate or increase to some beneficial degree, preferably to increase by at least about 30 percent, more preferably by at least 40 percent, more preferably by at least 50 percent, more preferably by at least 60 percent, more preferably by at least 70 percent, more preferably by at least 80 percent, most preferably by at least 90 percent, angiogenesis as compared to untreated controls.

The compounds and compositions disclosed herein may be administered by any route, including intradermally, subcutaneously, orally, intraarterially or intravenously.

The concentration of a disclosed compound in a pharmaceutically acceptable mixture will vary depending on several factors, including the dosage of the compound to be administered, the pharmacokinetic characteristics of the compound(s) employed, and the route of administration. Skilled workers can extrapolate the mouse data presented herein, which is based on 100 mg/kg, 0.1-1 μM plasma concentration, to reach the desired effect in the organism of interest. The agent may be administered in a single dose or in repeat doses.

As used herein “organism” refers to animals, preferably mammals, more preferably mammals such as experimental mammals or humans. Likewise the subject to be treated by the inventive methods can mean either a human or non-human animal.

As used herein, “vector” or “expression vector” refers to a nucleic acid construct, generated recombinantly or synthetically, with a series of specified nucleic acid elements that permit transcription of a particular nucleic acid in a host cell as known in the art. The expression vector can be part of a plasmid, virus, or nucleic acid fragment. Typically, the expression vector includes a nucleic acid to be transcribed operably linked to a promoter.

### BRIEF DESCRIPTION OF THE DRAWING FIGURES

FIG. 1 presents a sampling of gamma-secretase pathway inhibitors, where compounds in (a) are benzodiazepine and caprolactam group inhibitors, compounds in (b) are sulphonamides, compounds in (c) are dipeptides and semipeptides, compounds in (d) are benzodiazepines, and compounds in (e) are benzocaprolactams;

FIG. 2 shows results of cluster sprout length measurements after DAPT treatment;

FIG. 3 shows results of cluster sprout length measurements after DAPT and VEGF treatment;

FIG. 4 shows results of cluster sprout length measurements after 1-(S)-endo-N-(1,3,3)-Trimethylbicyclo[2.2.1]hept-2-yl)-4-fluorophenyl Sulfonamide treatment;

FIG. 5 shows results of cluster sprout length measurements after 1-(S)-endo-N-(1,3,3)-Trimethylbicyclo[2.2.1]hept-2-yl)-4-fluorophenyl Sulfonamide and VEGF treatment;

FIG. 6 shows results of cluster sprout length measurements after WPE-III-31C treatment;

FIG. 7 shows results of cluster sprout length measurements after WPE-III-31C and VEGF treatment;

FIG. 8 shows results of cluster sprout length measurements after Compound X treatment;

FIG. 9 shows results of cluster sprout length measurements after Compound X and VEGF-A treatment;

FIG. 10 shows results of cluster sprout length measurements after LY-450,139 treatment;

FIG. 11 shows results of cluster sprout length measurements after LY-450,139 and VEGF-A treatment;

FIG. 12 shows the central region of a control mouse retina;

FIG. 13 shows the central region of a treated mouse retina;

FIG. 14 shows the capillary enclosed areas of a control mouse retina;

FIG. 15 shows the capillary enclosed areas of a treated mouse retina;

FIG. 16 depicts quantitative data on retinal vessel density;

FIG. 17 shows labeled astrocytes in a control mouse retina;

FIG. 18 shows labeled astrocytes in a treated mouse retina;

FIG. 19 shows a retinal cross section with asterisks marking vascular tufts;

FIG. 20 shows vascular tufts in a control mouse retina;

FIG. 21 shows the absence of vascular tufts in a treated mouse retina;

FIG. 22 depicts quantitative data on retinal vascular tuft formation;

FIG. 23 depicts quantitative measurements of VEGF-A levels in mouse

FIG. 24 shows vascularization of a control mouse retina;

FIG. 25 shows vascularization of a control mouse retina;

FIG. 26 shows vascularization of a treated mouse retina.

FIG. 27 shows vascularization of a treated mouse retina.

FIG. 28a and b contain chemical formulas referred to in the background of the invention.

### DETAILED DESCRIPTION

Treatment with a gamma-secretase inhibitor surprisingly leads to decreased pathological angiogenesis and increased physiological angiogenesis. Such a treatment can normalize the angiogenic response and can alter the quality of the angiogenic response. While the novel use of gamma-secretase inhibitors to initiate or increase angiogenesis is a significant teaching of the present invention, this knowledge also helps further advance medicine in fields where inhibition of both gamma-secretase and angiogenesis is desired.

The examples described below employ cells or tissues from various sources, such as humans, based on availability, applicability to the materials being evaluated, and convenience. One skilled in the art will recognize that the inventive methods and compositions are also applicable to other mammals. This flexibility in applicability of results is supported by numerous sources, for example, murine models have been extrapolated to Alzheimer's in humans. C. elegans and Drosophila species have been used to elucidate Alzheimer's related pathways with good reproducibility in mice. The close homology between mammalian genes when compared to the non-mammal models is further evidence that the data from one species of mammal is applicable to other mammals. The same is also true with regard to the observations with regard to the gamma-secretase pathway.

It is accepted that oxygen-induced retinopathy models can be used to evaluate disease progression and the effect of different substances on AMD and diabetic retinopathy. Such a model is thus relied upon as described below. It has further been shown that observations related to retinal angiogenesis can be extended to angiogenesis in other tissues as well. See, for example, the discussion of two different drugs which work by targeting VEGF-A and their uses in AMD and colorectal cancer, respectively (Luttun et al., Nat. Med. August 2002; 8(8):831-40). This reference shows that Placenta Growth Factor (PIGF) can promote angiogenesis in both the heart and in skeletal muscle and that inhibition of PIGF inhibits angiogenesis in the ischemic retina, in tumors and in inflammation found with in autoimmune arthritis. Therefore, novel treatments and effects discussed below apply to angiogenesis generally.

The present disclosure that the gamma-secretase pathway and factors active therein influence angiogenesis forms the basis for treatment methods of many human and animal diseases. The invention also encompasses kits and reagents adapted to the subject methods.

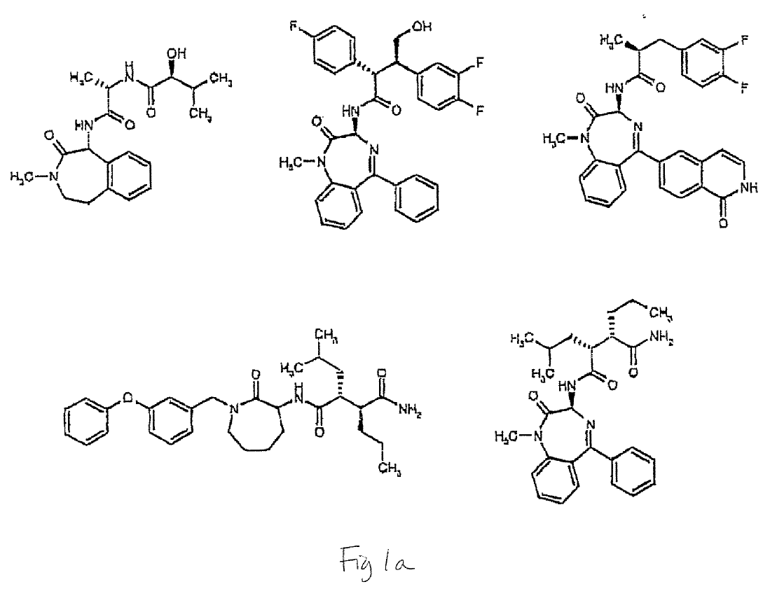

A person of ordinary skill in the art will readily recognize that a large number of potential gamma-secretase pathway inhibitors are already available. Examples of known inhibitors are shown in FIG. 1. According to embodiments herein, DAPT, 1-(S)-endo-N-(1,3,3)-Trimethylbicyclo[2.2.1]hept-2-yl)-4-fluorophenyl Sulfonamide, WPE-III-31C, (N)-[(S)-2-hydroxy-3-methyl-butyryl]-1-(L-alaninyl)-(S)-1-amino-3-methyl-4,5,6,7-tetrahydro-2H-3-benzazepin-2-one, and S-3-[N′-(3,5-difluorophenyl-alpha-hydroxyacetyl)-L-alanilyl]amino-2,3-dihydro-1-methyl-5-phenyl-1H-1,4-benzodiazepin-2-one are used as inhibitors of the gamma-secretase pathway. Additional inhibitors of the gamma-secretase pathway can be identified. For example, medicinal and combinatorial chemistry methods well-known to those skilled in the art can be used to modify known PS-1 antagonists to form new gamma-secretase inhibitors with improved efficacy for the purposes of the present invention. Further, as the above-named compounds have already been identified as useful compounds according to the invention, analogs of the same may be used.

In order to evaluate the efficacy of the gamma-secretase inhibitors for the purposes of the present invention, a variety of assays are conducted to evaluate the ability of the gamma-secretase inhibitor to initiate or increase angiogenesis. Examples of assays are well-known to those of ordinary skill in the art, see e.g. Murray, Angiogenesis protocols, in Murray, Methods in Molecular Medicine, 2001, ISBN 0-89603-687-7. Known gamma-secretase inhibitors rely on the previously-elucidated understanding of the role of the gamma-secretase pathway, making some inhibitors more and some inhibitors less effective at influencing angiogenesis. Accordingly, known factors may also be evaluated for their ability to create the results desired for the novel application disclosed herein.

Assays for a gamma-secretase inhibitor that creates the desired effect on angiogenesis may also rely on its role on tumor growth. Cell lines or animal models with a known propensity for tumorogenesis can be subjected to treatment with a candidate inhibitor. Tumor growth can be monitored and evaluations can be made of vascular parameters in the tumor and vascular density and morphology in biopsies from the tumor. These results can be compared with known or control values to indicate the efficacy of the gamma-secretase inhibitor/angiogenesis increaser tested.

A skilled worker could utilize materials in the art to determine how inhibitors, thus created, could be most effectively administered. Administration may preferentially be oral. Parenteral administration could also be utilized, particularly where the properties of the gamma-secretase inhibitor and any vehicles or diluents employed are not compatible with oral uptake and distribution. Dosing of gamma-secretase inhibitors would be based on the pharmacology of the inhibitor or inhibitor mixture, including consideration of IC50 values, metabolism, excretion and toxicity values. Administration may be for the purpose of managing disease, treating disease, preventing disease, research or other purposes. Examples of administration exist in the art, see, for example Siemers et al., Effect of LY450139, a functional gamma-secretase inhibitor, on plasma and cerebrospinal fluid concentrations A-beta and congnitive functionoing in patients with mild to moderate Alzheimer's Disease, Neurology, 2004:62 (supp 15).

The findings of the present invention are particularly useful in modulating conditions characterized by systemic or local abnormalities in angiogenic activity. Examples include, but are not limited to, eye diseases (e.g., AMD, retinopathy of prematurity, diabetic retinopathy), responses to organ transplantation, coronary artery disease, ischemic heart disease, wound healing, peripheral vascular disease, tumorogenesis/cancer, and inflammatory conditions (e.g., rheumatoid arthritis).

Specifically, disease states that are related to abnormal angiogenesis and could therefore be influenced with gamma-secretase inhibitors according to the present include atherosclerosis, hemangioma, hemangioendothelioma, vascular malformations, warts, pyogenic granulomas, hair growth, Kaposi's sarcoma, scar keloids, allergic edema, neoplasms, psoriasis, decubitus or stasis ulcers, gastrointestinal ulcers, dysfunctional uterine bleeding, follicular cysts, ovarian hyperstimulation, endometriosis, neoplasms, preeclampsia, placental insufficiency, respiratory distress, ascites, peritoneal sclerosis, adhesion formation, metastatic spreading, coronary artery disease, ischemic heart disease, ischemic limb disease, obesity, rheumatoid arthritis, synovitis, bone destruction, cartilage destruction, osteomyelitis, pannus growth, osterphyte formation, cancer, aseptic necrosis, impaired fracture healing, hepatitis, pneumonia, glomerulonephritis, asthma, nasal polyps, liver regeneration, pulmonary hypertension, systemic hypertension, diabetes, retinopathy of prematurity, diabetic retinopathy, choroidal disorders, intraocular disorders (e.g. age related macular degeneration), leukomafacia, stroke, vascular dementia, disease, thyroiditis, thyroid enlargement, thyroid pseudocyst, tumor metastasis, lymphoproliferative disorders, lympgoedema, AIDS, and hematologic malignancies.

Methods are available for monitoring the gamma-secretase pathway, which can facilitate, inter alia, analysis of the efficacy of inhibitors. For example, monitoring activity of the gamma-secretase pathway could be accomplished by creating a substrate for the gamma-secretase that can be detected in various assays. For example, Kinoshita et al. (2002, J Neurochem. 82:839-47) describes that the gamma secretase-generated carboxyl-terminal domain of APP (APP-CT) interacts in the cytoplasm with an adapter protein, Fe65, and this CT domain, when tagged with green fluorescent protein (GFP), may serve as a readout for processes that modify gamma secretase release of the APP-CT. APP-CT, when stabilized by FE65, translocates to the nucleus in a manner dependent upon stabilization by the adapter protein Fe65, and this translocation may be observed with laser scanning confocal microscopy. The APP-CT domain continues to interact with Fe65 in the nucleus, as determined by both colocalization and fluorescence resonance energy transfer (FRET). Alternatively, BRET2 (Bioluminescence Resonance Energy Transfer), as available commercially from Perkin Elmer, Torrance, Calif., or ELISA assays detecting processed APP fragments (see WO02/40451) may be used.

In a preferred embodiment, a fluorescent dye and a quencher are attached to either side of the gamma-secretase substrate cleavage site of a gamma-secretase substrate. When the substrate is intact, the dye and quencher are in close proximity and no signal is produced from the assay. When the substrate is cleaved the quencher is removed from the dye, a signal results which can be monitored and quantified. This assay could be performed as an isolated biochemical assay in vitro, in cells and in animal models in vivo. An alternate assay that could be used to monitor the efficacy of the drug in cells, animal models or human patients involves taking biopsies from diseased tissue and monitoring the cleavage of gamma-secretase substrates using conventional techniques such as detecting the presence and quantities of the substrates with antibodies.

Through research on malignant tumors, it has been found that certain tumors generate both angiogenesis-stimulating and inhibiting factors. This indicates that the angiogenic phenotype is the result of a balance between these positive and negative regulators of neovascularization. Novel means to increase angiogenesis may therefore be useful in conditions where systemic increased angiogenesis is disfavored.

In light of the present inventive disclosure, numerous new methods can be developed. These methods can be based on the knowledge that gamma-secretase inhibitors, such as DAPT, 1-(S)-endo-N-(1,3,3)-Trimethylbicyclo[2.2.1]hept-2-yl)-4-fluorophenyl Sulfonamide, WPE-III-31C, (N)-[(S)-2-hydroxy-3-methyl-butyryl]-1-(L-alaninyl)-(S)-1-amino-3-methyl-4,5,6,7-tetrahydro-2H-3-benzazepin-2-one, and S-3-[N′-(3,5-difluorophenyl-alpha-hydroxyacetyl)-L-alanilyl]amino-2,3-dihydro-1-methyl-5-phenyl-1H-1,4-benzodiazepin-2-one initiate and increase angiogenesis. Certain of the new methods of the invention rely on comparisons of model systems' reactions to treatment with a gamma-secretase inhibitor relative to treatment with a control or no treatment. It is understood that practices commonly-used in the art are to be followed, for example, that except for the administration of gamma-secretase inhibitor the test conditions are otherwise as equivalent as possible. Certain other of the novel methods rely on evaluating one or more aspects or activities of gamma-secretase or the gamma-secretase activity in a model system. The skilled worker would determine which of these parameters to evaluate in order to best perform the novel methods. The model systems used may include any suitable model, such as the Oxygen-induced Retinopathy Model (see, Smith et al., Invest Ophtalmol Vis Sci 35, 101-11 (1994)).

Experimentation and analysis conducted during the pursuit of the present invention are described below as particular examples but not by way of limitation. Alternate methods known to skilled workers are within the scope of the invention. Unless otherwise noted, materials and equipment described herein are commercially available.

### EXAMPLE 1

### In vitro Model for Analysis of Gamma-Secretase Inhibitors

Human umbilical vein endothelial cells (HUVEC) were used to evaluate various gamma-secretase inhibitors for potential angiogenesis effect. A 72 cm2 flask of confluent cells (1.5-2 E6 cells) was trypsinized with 3.5 ml trypsin/EDTA. After a maximum of 3 minutes, the cells were rinsed with 1.5 ml serum then centrifuged at 200 g for 3 minutes. After centrifugation, the supernatant was discarded and the cells resuspended in medium (endothelial cell growth medium (Promocell) with additional 10% serum). Cell concentration was diluted to 2.0×104 cells/ml in medium supplemented with 20% methocel stock solution. The cells were clustered in hanging drops of 20 μl (400 cells) overnight.

To imbed the cells in gel, the drops were first rinsed with PBS. The clusters were centrifuged at 200 g for 3 minutes and the supernatant removed. Centrifuge tubes were then briefly drawn over a rough surface to loosen the pellet. The clusters were resuspended in ice cold methocel stock solution supplemented with 20% protein. An equal amount of collagen I-gel at 3 mg/ml (Becton-Dickinson) was carefully mixed with the cells. Immediately thereafter, 1000 μl of the gel/cell cluster mixture was added to each well of a preheated (37° C.) 24-well plate. The plate was incubated for 30 minutes at 37° C., 5% CO2 and 100% humidity. One hundred pi of medium, along with the desired test substance and optionally 2.5 μl of 10 μg/mi VEGF were added to each well to a final concentration in the top medium of 250 ng/ml and 25 ng/ml in the well. Negative control cells received medium only and positive control cells received medium and VEGF only. The wells without gel were given 1 ml PBS.

To quantify the results, ten clusters from each cell were photographed. The length of the five longest sprouts on each cluster was measured, and the sum of the sprout length from each cluster calculated. Average values from ten clusters were obtained.

The above-mentioned methocel stock solution was prepared by autoclaving 6 grams of pure methyl cellulose powder (Sigma) in a 500 ml flask containing a magnetic stirrer. After autoclaving the methyl cellulose was dissolved in 250 ml of basal medium preheated to 60° C. for 20 minutes. An additional 250 ml of basal medium at room temperature was added to a final volume of 500 ml and the solution stirred overnight. Finally, the stock solution was aliquotted and cleared through centrifugation at 5000 g for 2 hours at room temperature. The 90-95% of the material which became the clear, highly viscous supernatant was used.

### EXAMPLE 2

### Treatment with DAPT

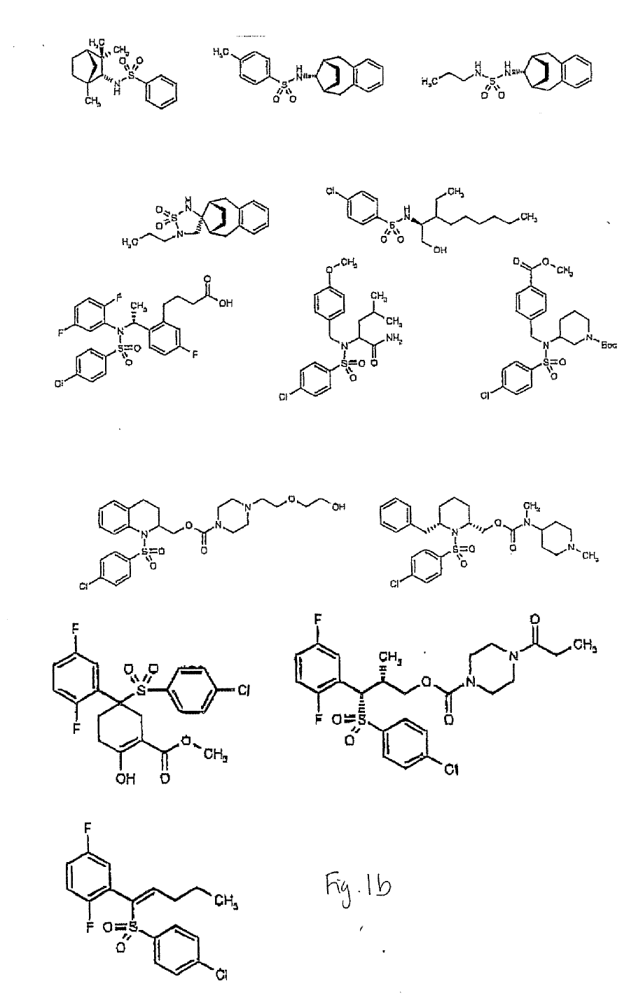

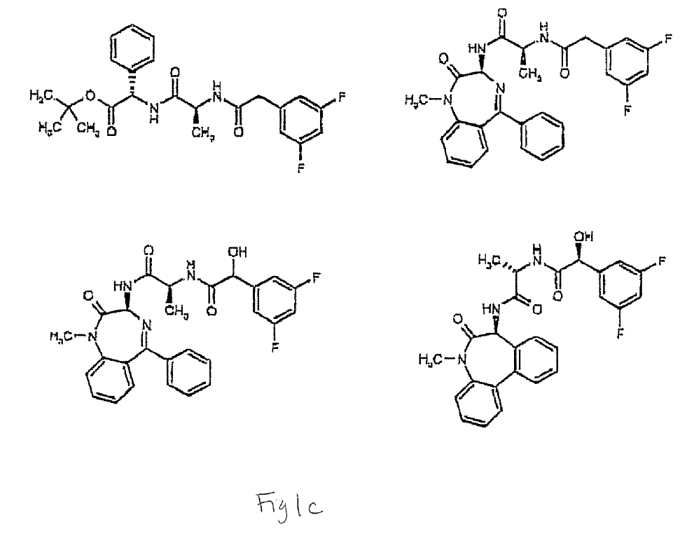

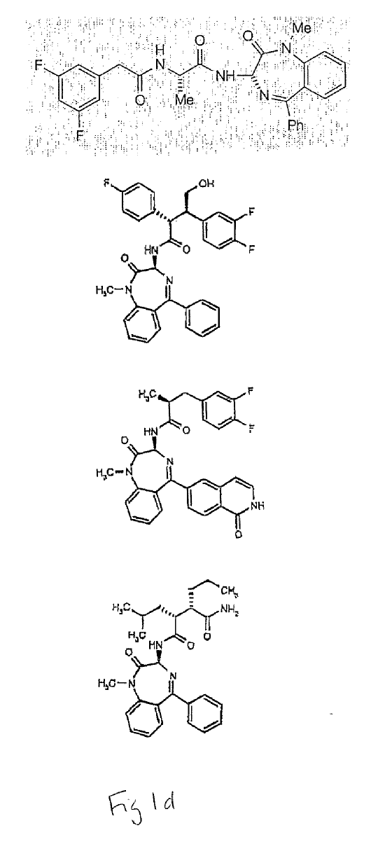

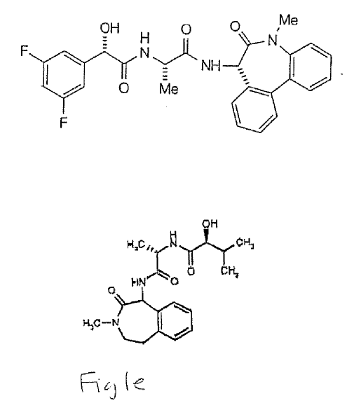

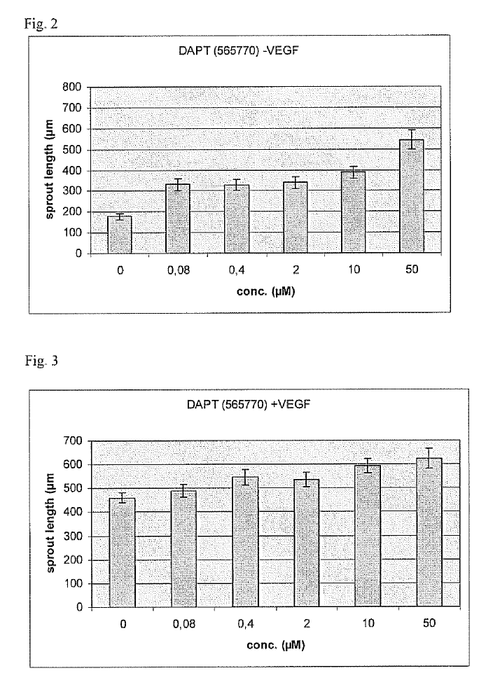

Using the above procedure, cells were treated with varying amounts of DAPT (stock solution 5 mM DAPT in DMSO; Calbiochem cat. no. 565770). Some of the cells were treated with both DAPT and VEGF. Results are shown in FIGS. 2 and 3. As can be seen from the figures, compounds of the gamma-secretase inhibitor dipeptide class such as DAPT promote angiogenesis. The data in FIG. 2 shows that cells treated with neither DAPT nor VEGF had an average sprout length of 180 μm±16 μm (SEM). Cells treated with 0.08 μM DAPT had a significant increase in sprout length, to 330±28 μm p-value <0.001 (one-sided paired t-test). Increasing levels of DAPT to 0.4, 2,10 and 50 produced further increases in sprout length.

Where VEGF was co-administered with DAPT, the sprout length was even longer, see FIG. 3. Control cells receiving VEGF only had an average sprout length of 460 μm±20 μm. Those treated with DAPT as well showed increases in sprout length over those with VEGF alone, although the increase was not as marked as in the cells treated with DAPT alone. Cells treated with 0.4 μm DAPT has a significant increase in sprout length, to 550±32 μm p-value<0.01 (one sided paired t-test).

### EXAMPLE 3

### Treatment with 1-(S)-endo-N-(1,3,3)-Trimethylbicyclo[2.2.1]hept-2-yl)-4-fluorophenyl Sulfonamide

Using the procedure of Example 1, 0.16 μM and 0.8 μM concentrations of sulfonamide class gamma-secretase inhibitor (Calbiochem cat. no. 565763) were evaluated along with control cells for sprout length. As seen in FIG. 4, the control cells had an average length of 120 μm±15 μm whereas the cells treated with 0.16 μM 1-(S)-endo-N-(1,3,3)-Trimethylbicyclo[2.2.1]hept-2-yl)-4-fluorophenyl Sulfonamide had an average length of 280 μm±23 μm, p-value<0.001. This 100% increase in length shows that sulfonamide-class gamma-secretase inhibitors promote angiogenesis. When co-administered with VEGF, a concentration of 20 μM 1-(S)-endo-N-(1,3,3)-Trimethylbicyclo[2.2.1]hept-2-yl)-4-fluorophenyl Sulfonamide resulted in an average sprout length of 480 μm±19 μm, whereas control cells treated with VEGF alone had an average length of 370 μm±25 μm, p-value<0.001. While not wanting to be bound by theory, more titrations might reveal effects of a Sulfonamide group compound at lower concentrations as well.

### EXAMPLE 4

### Treatment with WPE-III-31C

Using the procedure of Example 1, a transition state mimic gamma-secretase inhibitor (Calbiochem cat. no. 565778) was evaluated for its ability to promote angiogenesis, see FIGS. 6 and 7. In addition to control cells, 0.16 μM, 0.8 μM and 4 μM concentrations were evaluated. While the average sprout length for control cells was only 140 μm±22 μm, that for the lowest concentration of WPE-III-31C tested was 230 μm±24 μm, p-value<0.01. The sprout length only increased with increasing concentrations of WPE-III-31C, demonstrating its utility as an angiogenesis promoter.

When the compound was co-administered with VEGF, cells receiving WPE-III-31C showed significantly longer sprout lengths than the control cells, which received only VEGF. While the average sprout length for control cells was only 320 μm 18 μm, that for the lowest concentration (0.16 μM) of compound tested was 410 μm±17 μm, p-value<0.001. This reinforces the findings shown in FIG. 6, that is, the angiogenesis-promoting activity of transition state mimic gamma-secretase inhibitors.

### EXAMPLE 15

### Modified in vitro Model for Analysis of Gamma-Secretase Inhibitors

Similar to the approach described in Example 1, two further compounds were evaluated for their ability to initiate or increase angiogenesis. HUVEC spheroids were prepared by pipetting 400 HUVE cells (PromoCell, Heidelberg, Germany, cultured according to supplier's instructions) into each well of non-adhesive 96-well plates and allowed to aggregate overnight. After harvesting, 48 spheroids were seeded in 900 μl of methocel-collagen solution and pipetted into individual wells in a 24-well plate, allowing for collagen gel polymerization. After 30 minutes, freshly-prepared test compounds were added (prepared by dissolving lyophilized compound at a concentration of 10 mM in DMSO, specific concentrations administered described below) by pipetting 100 μl of a 10-fold concentrated working dilution on top of the gel. At the end of the incubation period the dishes were fixed for 24 hours at 37° C. with paraformaldehyde.

Sprouting intensity of endothelial cells was quantitated by an image analysis system determining the cumulative sprout length per spheroid using an Olympus IX50 inverted microscope and Olympus image analysis software. The mean of the cumulative sprout length of 10 randomly selected spheroids was analyzed as an individual data point.

### EXAMPLE 16

### Treatment with Compound X

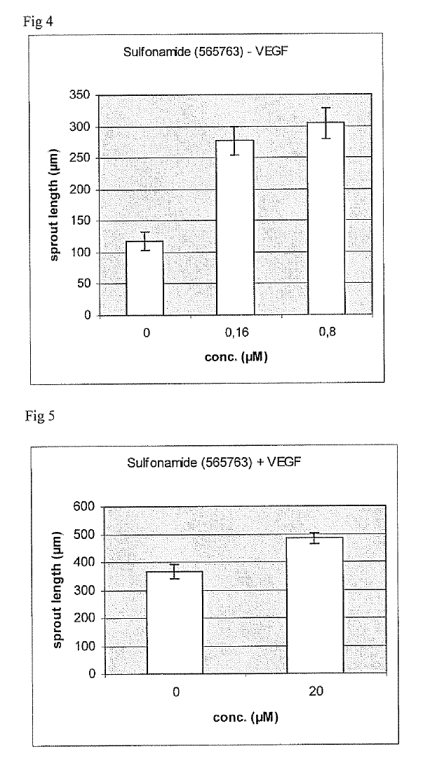

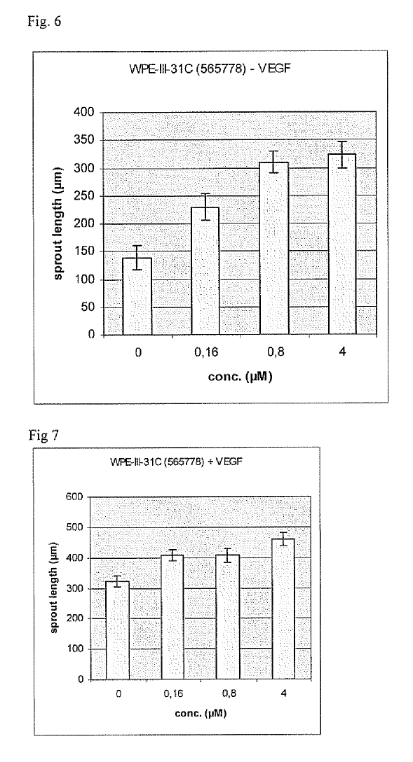

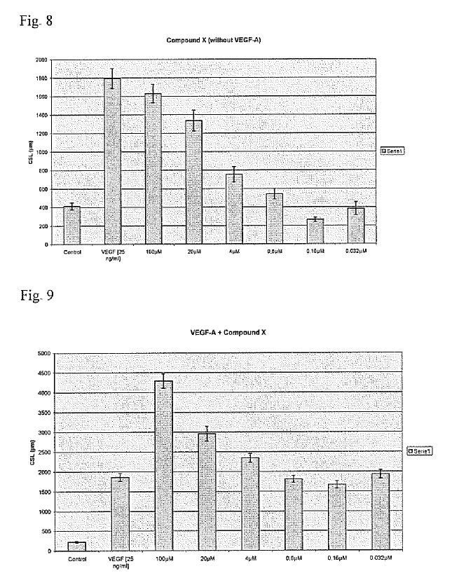

Using the procedure of Example 5, a benzodiazepine class gamma-secretase inhibitor (prepared according to WO98/28268 which is expressly incorporated by reference herein) was evaluated for its ability to promote angiogenesis, see FIGS. 8 and 9. In addition to control cells and cells receiving 25 ng/ml VEGF, six different concentrations were evaluated: 100 μM, 20 μM, 4 μM, 0.8 μM, 0.16 μM, 0.032 μM. In this case sprout length was evaluated by quantifying the cumulative length of all sprouts (CSL). While the CSL for control cells was only 413 μm±37 μm, the 100 μM concentration of Compound X produced an CSL of 1630 μm±99 μm. This was slightly lower than the VEGF-A treated sprouts which reached 1794 μm±107 μm. At decreasing compound concentrations the measured sprout length decreased, with the exception of the 0.032 μM group which had a slightly higher result than the 0.16 μM group. Only the two lowest concentrations tested failed to produce longer sprouts that control. The increase in sprout length as compared to the control, particularly at higher concentrations, demonstrates the utility of Compound X as an angiogenesis promoter, the 100 μM concentration being nearly as effective as VEGF-A.

When Compound X was co-administered with VEGF-A, cells showed significantly longer sprout lengths than the control cells or the VEGF-A-only cells. At the 100 μM concentration, the CSL was 4289 μm±183 μm as compared to only 223 μm±24 μm, a remarkable increase. While the CSL is usually about 200 μm, higher control results can be attributed to internal experimental variability. The lower doses of test compound may appear to produce less significant stimulatory results because of the slightly high control values. That said, even the addition of 4 μM Compound X was enough to increase the sprouting significantly. This reinforces the findings shown in FIG. 8, that is, the angiogenesis-promoting activity of benzodiazepine class gamma-secretase inhibitors.

### EXAMPLE 7

### Treatment with LY-450, 139

Using the procedure of Example 5, a benzocaprolactam class gamma-secretase inhibitor (prepared according to WO02/47671, WO02/40508 and WO02/40451 which are expressly incorporated by reference herein) was evaluated for its ability to promote angiogenesis, see FIGS. 10 and 11. In addition to control cells, and cells receiving VEGF-A, six different concentrations were evaluated: 100 μM, 20 μM, 4 μM, 0.8 μM, 0.16 μM, 0.032 μM. Sprout length was evaluated by quantifying the cumulative length of all sprouts. While the average sprout length for control cells was 413 μm±37 μm, the 100 μM concentration of the compound evaluated produced an average sprout length of 1730 μm±95 μm. At decreasing compound concentrations the measured sprout length typically decreased. While the lower concentrations were not found to be effective, at the higher concentrations tested, LY-450,139 demonstrated utility as an angiogenesis promoter.

When LY-450,139 was co-administered at a concentration of 25 ng/ml VEGF-A, cells showed significantly longer sprout lengths than the control cells, 3521 μm±229 μm for the 100 μM dose group as compared to only 223 μm±24 μm in the control and 1860±100 μm for VEGF-A, a remarkable increase. Only the lowest concentration tested failed to produce longer sprouts that VEGF-A, and all produced considerably longer sprouts than control. FIGS. 10 and 11 display the applicability of benzocaprolactam class gamma-secretase inhibitors as angiogenesis promoters.

### EXAMPLE 8

### DAPT Evaluations in vivo

Oxygen-induced retinopathy mice were used to determine the extent and effect of gamma-secretase inhibitors on the angiogenic process. Mice with oxygen induced retinopathy (OIR mice) are a commonly-used model and are described in the literature (see, Smith et al, supra). They are at times referred to as retinopathy of prematurity models or ROP mice. The model takes advantage of the fact that full term mice pups are born with an immature retinal vascularization which matures during the first three weeks of postnatal growth.

Briefly, neonatal mice (NMRI/C57b1) from the same litter were placed, with their nursing mother, in a hyperoxic environment (75% oxygen) at age day seven. After exposure to the hyperoxic environment for 5 days, the 12-day old pups were removed to normal air. In the treated mice, DAPT was administered once daily during age days 12-16. Control pups were treated the same way but injected with the vehicle. At post natal day 17 the mice were euthanized and the retinas were prepared for whole mount immunohistochemistry.

Preparation and Administration of DAPT

Except for the mode of injection, the stock solution of DAPT was prepared and administered essentially as described in Lanz et al., (2003, J. Pharmacol. Exp. Ther. 305:864-71). In short, 5 mg DAPT was dissolved in 25 μl 99.5% EtOH and then dissolved in 475 μl Corn oil (Sigma, Catalog No. C8267). If precipitate formed, the solution was heated to 70° C. for 2-3 minutes. The pups were injected subcutaneously once a day according to: V (μl)=weight (g)*10, that is, 100 mg DAPT/kg body weight. Control pups were injected subcutaneously once a day with the same amount of vehicle (i.e. 5% EtOH in corn oil).

Whole Mount Immunohistochemistry

Eyes were fixed in 4% PFA in PBS at 4° C. overnight and washed in PBS. Retinas were dissected, permeabilized in PBS, 1% BSA, and 0.5% TritonX-100 at 4° C. overnight, rinsed in PBS, washed twice in PBlec (PBS, pH 6.8, 1%Triton-X100, 0.1 mM CaCl, 0.1 mM MgCl, 0.1 mM MnCl) and incubated in biotinylated isolectin B4 (Bandeiraea simplicifolia; L-2140; Sigma-Aldrich) 20 g/ml in PBlec at 4° C. overnight. After five washes in PBS, samples were incubated with streptavidin conjugates (Alexa 488, 568, or 633; Molecular Probes) diluted 1:100 in PBS, 0.5% BSA, and 0.25% Triton X-100 at 4° C. for 6 hours. TO-PRO 3 (1:1,000; Molecular Probes) served for nuclear staining. After washing and a brief post fixation in PFA, the retinas were flat mounted using Mowiol/DABCO (Sigma-Aldrich).

As further described below, normal mice pups at 17 post natal days after oxygen induced retinopathy exhibit formation of avascular zones in the central, i.e., close to the optic nerve, areas of the retina. At the same time there is an increased vascular density in the peripheral parts of the retina. This can be quantified in the superficial capillary plexus by counting the number of capillary enclosed areas in the peripheral part of the retina.

### EXAMPLE 9

### Evaluation of Retinal Vascularization and Vessel Density in Gamma-Secretase Inhibitor Treated OIR Mice

Avascular Retinal Space

By comparing the retinas of control pups to those pups treated with gamma-secretase inhibitor, the effect of the inhibitor on angiogenesis was evaluated. As previously stated, otherwise untreated OIR mice pups will exhibit avascular zones in the central retina. FIG. 8 shows the avascular space (avascular zones marked AZ in the figure) of a normal OIR mouse retina. In a surprising contrast, DAPT-treated OIR mice exhibited central retinal vascularization as shown in FIG. 9. The treated mice almost completely lack the vascular-free zones. This is one example of the angiogenesis-initiating effect of gamma-secretase inhibitors.

Peripheral Retina Vascular Density

Furthermore, at 17 post natal days after OIR, there is an increased vascular density in the peripheral parts of the retina. This can be quantified in the superficial capillary plexus by counting the number of capillary enclosed areas in the peripheral part of the retina. FIG. 10 shows a control mouse retina, with asterisks pointing out capillary enclosed areas. The peripheral retina shown in FIG. 11 is from a DAPT-treated mouse, showing a significant increase in capillary enclosed areas and therefore an angiogenesis-increasing effect. To further evaluate these results, the capillary enclosed areas were counted. The data presented in FIG. 12 (p<0.001) reflects the near doubling of such areas in DAPT-treated mice.

Astrocyte Interaction

Because retinal vessels grow in tight interaction with astrocytes, changes in the astrocytic network can lead to changes of the vasculature. To rule out the possibility that the observed increase in vessel density was due to an increased number of astrocytes, the retinas from control and DAPT-treated animals were stained with Glial Acidic Fibrillary Protein (GFAP) antibodies which specifically label astrocytes (DAKO). The GFAP antibodies were first diluted 1:75 and incubated at 4° C. overnight, after washing the tissue was incubated with secondary antibody (anti-rabbit-Cy3red,Novakemi 111 165 144) diluted 1:100. FIGS. 13 and 14 show the astrocytic network of control and DAPT-treated mouse retinas, respectively. Little if any difference was observed in the density of GFAP positive astrocytes, therefore no vascular changes could be attributed to changes in the astrocytic network.

### EXAMPLE 10

### Quantification of Angiogenesis in Gamma-Secretase Inhibitor Treated OIR Mice

Vascular Tufts

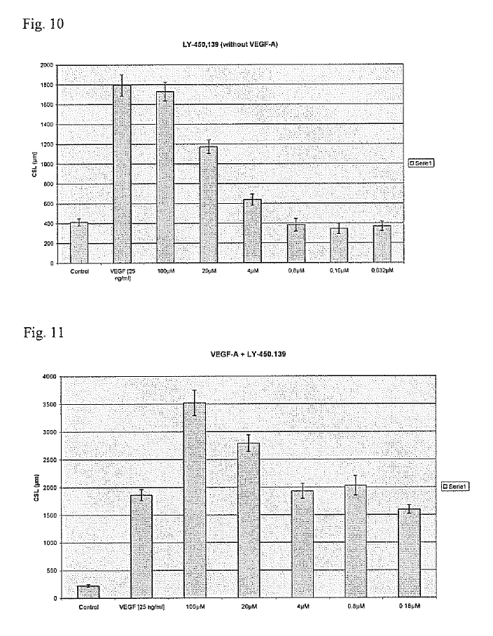

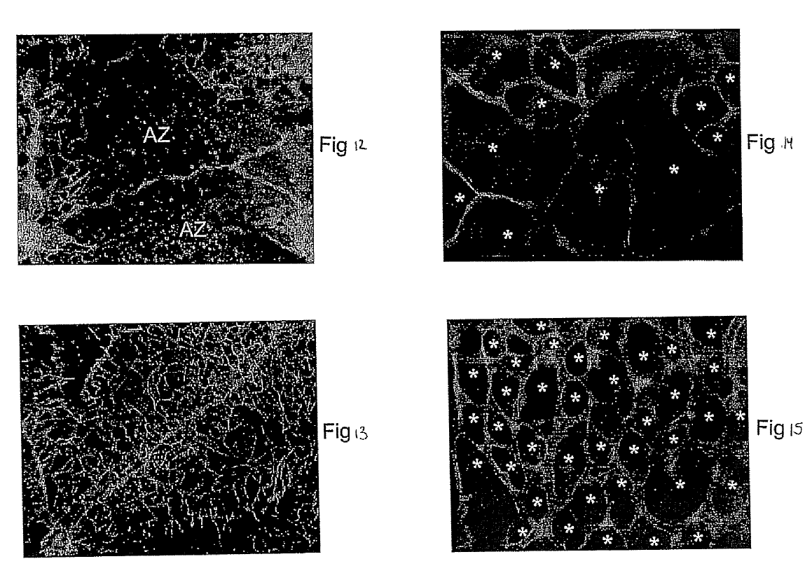

Vascular tufts form in the retinas of OIR mice models. The tufts consist of endothelial cells growing in a small localized cluster above the inner limiting membrane and pouching into the vitreous. FIG. 15 shows a retinal cross section with asterisks marking vascular tufts. FIG. 16 shows a normal OIR mouse retina and numerous vascular tufts. Despite the increased angiogenic response seen in DAPT-treated OIR mice, a significant reduction in capillary tufts was observed as shown in FIG. 17.

The numbers of capillary tufts can be quantified as a measurement of the pathological angiogenic response in the OIR model. This can be done, for example, with whole retinas stained with isolectin using a Nikon Microphot-FXA microscope and 4× magnification lens. Quantification of the results in this case are reflected in FIG. 18 (p-value<0.01).

### EXAMPLE 11

### Evaluation of Possible Role of Other Pro-Angiogenic Therapies in Gamma-Secretase Inhibitor Treated OIR Mice

VEGF-A (Vascular Endothelial Growth Factor A) is an important factor for both physiological and pathological angiogenesis and has been shown to be important for the vascular changes seen in association with OIR. Other pro-angiogenic therapies are known, see Example 13.

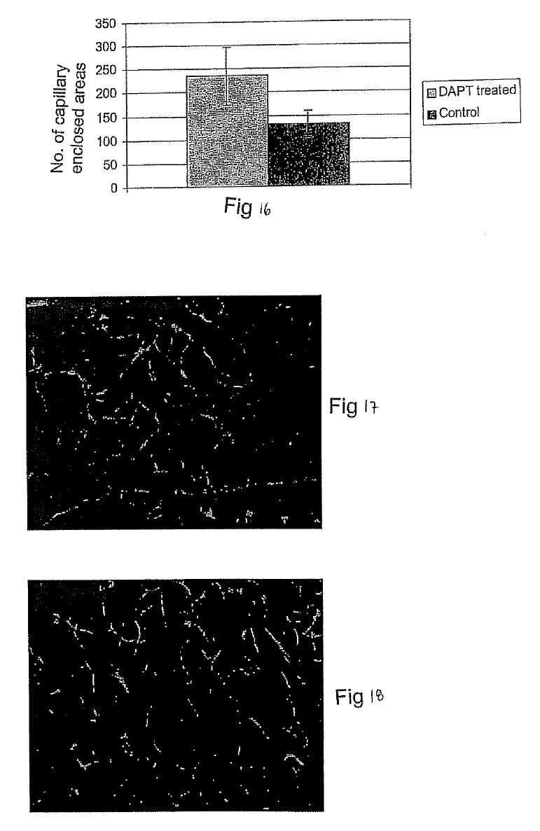

The increased vascularization in the DAPT-treated animals could potentially have been due to another pro-angiogenic factor such as up-regulation of VEGF-A. To evaluate whether VEGF-A was a factor in the surprising angiogenesis initiation and increase observed with administration of DAPT, the amount of VEGF-A was quantified using an ELISA detecting VEGF-A protein (R&D Systems, Minneapolis, Minn., USA). The protein was clearly detected in lysates of retina from both control and DAPT treated animals but no significant changes could be measured between the two groups. Supporting data are presented in FIG. 19.

### EXAMPLE

### Evaluation of Retinal Vessel Density and Architecture in Gamma-Secretase Inhibitor Treated Non-OIR Mice

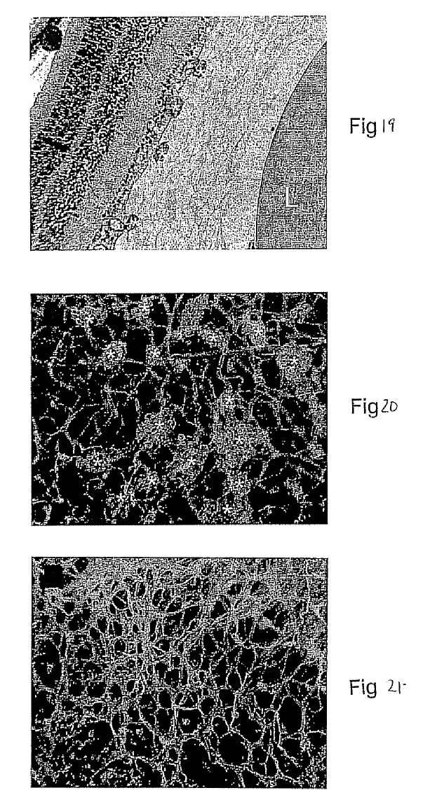

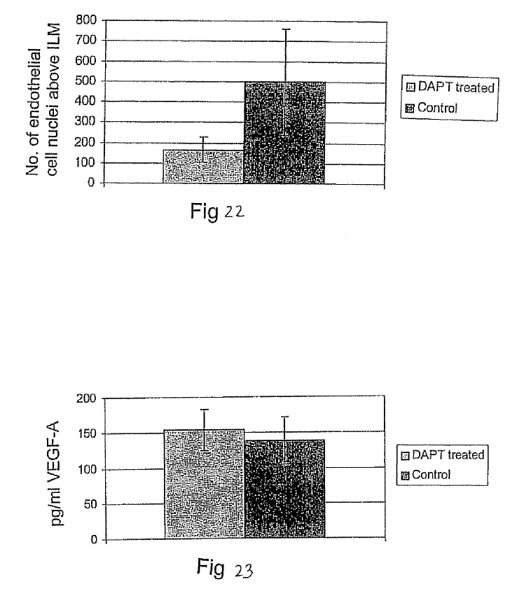

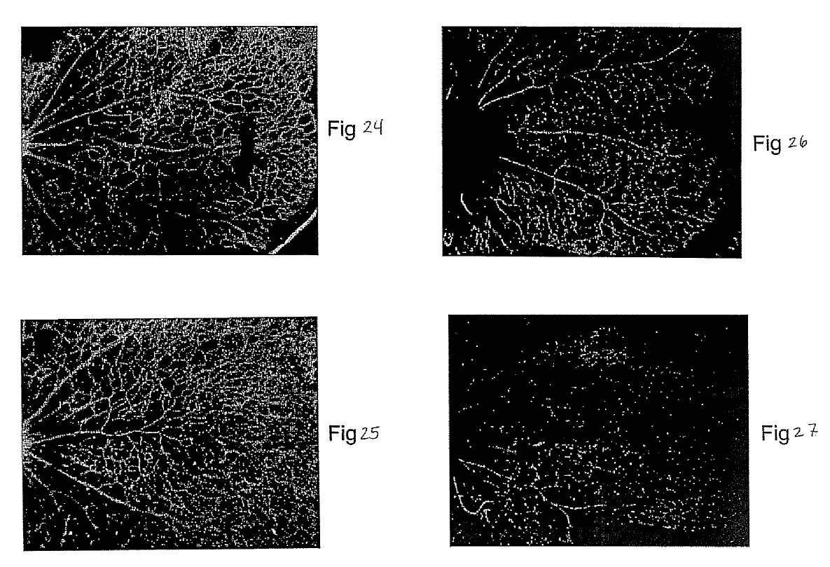

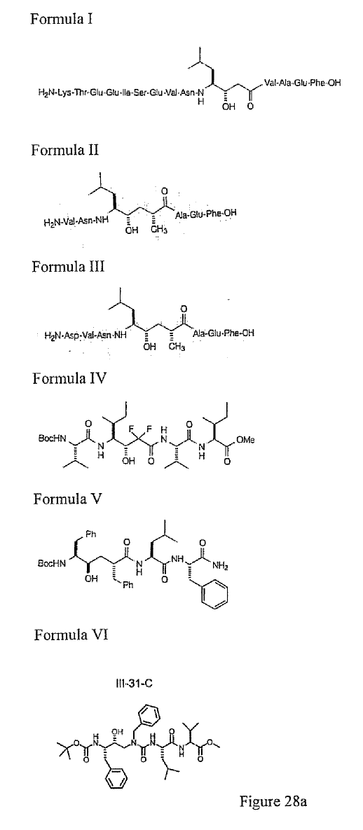

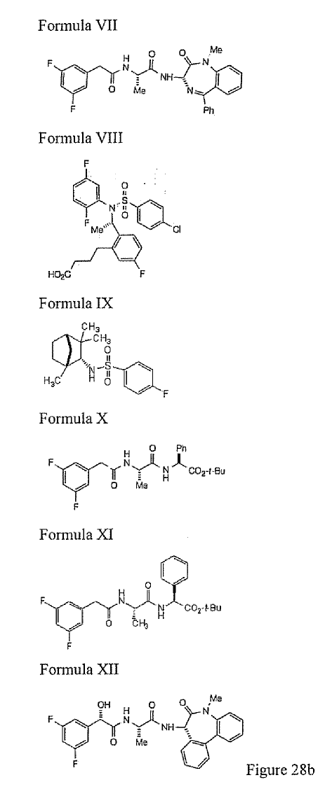

To investigate if the increased retinal vascularization after DAPT treatment was restricted to the OIR, the effect of DAPT on physiological angiogenesis in new born mice was investigated. DAPT was administered to new born mice on postnatal days 3 and 4 and retinas were analyzed at day 5 as described above. As was observed with OIR mice treated with DAPT, there was an increased peripheral vascular density. This can be observed by comparing the degree of vascularization in the retina of control animals, FIGS. 20 and 21, with that of treated animals as shown in FIGS. 22 and 23. Except for the increased vascular density, the vascular architecture was normal. The growth of the vascular network towards the periphery and the arterio-venous specification were intact for DAPT-treated mice as with controls.

### EXAMPLE 13

### Evaluation of Gamma-Secretase Inhibitor in Animal Models of Myocardial and Limb Ischemia, Myocardial Infarction, and Peripheral Ischemia

Mouse Models of Myocardial and Limb Ischemia

For therapeutic angiogenesis, a gamma-secretase inhibitor (e.g. DAPT) is delivered during the course of seven days to male Swiss mice aged 10-12 weeks. Thereafter, infarcted hearts are processed for morphometric analysis after immunostaining for endothelial thrombomodulin, which stains all vessels, or for smooth muscle α-actin, which stains mature SMC-covered vessels (see, for example, Lutgens et al., March 1999 Chronic myocardial infarction in the mouse: cardiac structural and functional changes, Cardiovasc Res.; 41(3): 586-93).

To induce limb ischemia, unilateral right or bilateral ligations of the femoral artery and vein, proximal to the popliteal artery, and the cutaneous vessels branching from the caudal femoral artery side branch are performed without damaging the nervus femoralis. Gamma secretase inhibitors can be administered as described above. Two superficial preexisting collateral arterioles, connecting the femoral and sphenoid artery, are used for analysis. Functional perfusion measurements of the collateral region can be performed using a Lisca PIM II camera (Gambro, Breda, the Netherlands) and analyzed (see, for example, Couffinhal, T. et al. 1998 Mouse model of angiogenesis., Am. J. Pathol. 152, 1667-1679). Perfusion, averaged from 3 images per mouse in the upper hind limb (adductor region where collaterals enlarge) or in total hind limb, is expressed as a ratio of right (ischemic) to left (normal) limb. Spontaneous mobility is scored by monitoring the gait abnormalities, the position of right foot in rest and after manipulation, and the “tail-abduction-reflex.” Mice are scored 0 when one observation is abnormal and 1 when normal. Based on the results of the present invention, it is expected that such models of myocardial and limb ischemia will reveal DAPT-treated mice exhibit increased angiogenesis resulting in increased perfusion and formation of collateral vessels leading to increased healing/decreased tissue damage and increased function of the tissue.

Endurance Exercise Swim Test for Mice

Mice are conditioned for 9 days to swim in a 31° C. controlled swimming pool under non-stressed conditions. At day 10, baseline exercise time for each mouse is determined using a counter-current swimming pool kept at 31° C.; flow at 0.2 m/s. For determining maximal endurance exercise, i.e., the total swimming period until fatigue, the failure to rise to the surface of the water to breathe within 7 seconds is assessed. At day 11, the femoral artery is occluded as described above. At day 18 minipumps are removed under isoflurane anesthesia before endurance exercise.

Recovery of functionality is expressed as a ratio to the baseline exercise time. Fluorescent microspheres (yellow-green, 15 μm, 1×106 beads per ml, Molecular Probes, Eugene, Oreg.) are administered after maximal vasodilation (sodium nitroprusside, 50 ng/ml, Sigma), processed, and flow calculated. Bismuth gelatino-angiography is performed and photo-angiographs (Nikon D1 digital camera) are analyzed in a blinded manner. Collateral side branches are categorized as follows: second-generation collateral arterioles directly branch off from the main collateral, whereas third-generation collateral arterioles are orientated perpendicularly to the second-generation branches. The number of collateral branches per cm length of the primary collateral arteriole is counted. Fluoroangiography is performed with a modified version of a described protocol (Carmeliet, P. et al., 2001 Synergism between vascular endothelial growth factor and placental growth factor contributes to angiogenesis and plasma extravasation in pathological conditions. Nature Med. 7, 575-583). Images are then reconstructed using, for example, a Zeiss LSM510 confocal laser microscope.

After perfusion-fixation, the 2 superficial collateral arterioles are post-fixed in paraformaldehyde 1% and paraffin-embedded. Twelve 5-μm cross-sections per superficial collateral, starting from the midzone and ranging over 1.95 mm to each end, are morphometrically analyzed. Collateral side branches are categorized as second generation (luminal area>300 μm2) or third generation (<300 μm2). Total perfusion area is calculated using the total sum of the side branch luminal areas. Capillary density is determined by immunostaining for thrombomodulin. Wall thickness of fully SMC-covered vessels is morphometrically measured on histological sections, after smooth muscle (X-actin staining. For all treatment groups, six cross-sections (150 μm apart) are analyzed per main collateral. Only second-generation collateral arterioles larger than 300 μm2 are included in the analysis. At least 10 measurements of wall thickness of the second-generation collateral arterioles are obtained. Based on the results of the present invention, it is expected that such endurance models will reveal DAPT-treated mice exhibit increased angiogenesis resulting in increased perfusion and formation of collateral vessels leading to increased healing/decreased tissue damage and increased function of the tissue.

### EXAMPLE 14

### Combination Therapies

The data provided above, particularly in Examples 2-4 and 6-7, demonstrate that gamma-secretase inhibitors represent a new and useful means of initiating or increasing angiogenesis. They also demonstrate that such inhibitors, when used in conjunction with a pro-angiogenic compound, have amplified effect. Thus, it is contemplated in the invention that gamma-secretase inhibitors will be combined with pro-angiogenics to provide superior results.

The beneficial combination of gamma-secretase inhibitor and VEGF-A was demonstrated above, however, other pro-angiogenic therapies are expected to be equally effective in enhancing the combinatorial effect of the two compounds. Gene therapies or protein based remedies may be the most suitable way to administer these materials. Examples of such pro-angiogenic therapies include growth factors such as VEGF-A, -B, -C, -D, FGF-1, -2, -4, HIFalpha and HGF.

Depending on the materials chosen and the condition to be affected, the two materials may be administered together in the same form, such as an injection. Alternatively, the administration may be in separate forms such as an injection and an oral composition. Such separate routes of administration may be simultaneous or successive. There may be a single or multiple administrations of one or both of the therapeutics, such as local delivery of a growth factor combined with systemic delivery of a gamma-secretase inhibitor.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the spirit and scope of the appended claims and equivalents thereof. The references disclosed herein, including U.S. patents, are each specifically incorporated by reference in their entirety. However, the citation of such references shall not be construed as an admission that the references are prior art to the present invention.

What is claimed is:

1. An angiogenesis initiator or increaser, comprising a pharmaceutically-effective amount of a gamma-secretase pathway inhibitor.

2. An angiogenesis initiator or increaser according to claim 1, wherein said gamma-secretase pathway inhibitor comprises a dipeptide class gamma-secretase pathway inhibitor.

3. An angiogenesis initiator or increaser according to claim 2, wherein said dipeptide class gamma-secretase inhibitor is DAPT (N-[N-(3,5-Difluorophenacetyl-L-alanyl)]-S-phenylglycine t-Butyl Ester).

4. An angiogenesis initiator or increaser according to claim 1, wherein said gamma-secretase pathway inhibitor comprises a sulfonamide class gamma-secretase inhibitor.

5. An angiogenesis initiator or increaser according to claim 4, wherein said sulfonamide class gamma-secretase inhibitor is 1-(S)-endo-N-(1,3,3)-Trimethylbicyclo[2.2.1]hept-2-yl)-4-fluorophenyl Sulfonamide.

6. An angiogenesis initiator or increaser according to claim 1, wherein said gamma-secretase pathway inhibitor comprises a transition state mimic class gamma-secretase inhibitor.

7. An angiogenesis initiator or increaser according to claim 6, wherein said transition state mimic class gamma-secretase inhibitor is WPE-III31C.

8. An angiogenesis initiator or increaser according to claim 1, wherein said gamma-secretase pathway inhibitor comprises a benzodiazepine class gamma-secretase pathway inhibitor.

9. An angiogenesis initiator or increaser according to claim 8, wherein said benzodiazepine class gamma-secretase inhibitor is S-3-[N′-(3,5-difluorophenyl-alpha-hydroxyacetyl)-L-alanilyl]amino-2,3-dihydro-1-methyl-5-phenyl-1H-1,4-benzodiazepin-2-one.

10. An angiogenesis initiator or increaser according to claim 1, wherein said gamma-secretase pathway inhibitor comprises a benzocaprolactam class gamma-secretase inhibitor.

11. An angiogenesis initiator or increaser according to claim 10, wherein said benzocaprolactam class gamma-secretase inhibitor is (N)-[(S)-2-hydroxy-3-methyl-butyryl]-1-(L-alaninyl)-(S)-1-amino-3-methyl-4,5,6,7-tetrahydro-2H-3-benzazepin-2-one.

12. A method of influencing a disease state in a cell, a group of cells, or an organism, comprising:
administering at least one of a gamma-secretase inhibitor or a gamma-secretase pathway inhibitor to the cell, group of cells, or organism,
wherein the disease is selected from the group consisting of atherosclerosis, hemangioma, hemangioendothelioma, vascular malformations, warts, pyogenic granulomas, hair growth, Kaposi's sarcoma, scar keloids, allergic edema, neoplasms, psoriasis, decubitus or stasis ulcers, gastrointestinal ulcers, dysfunctional uterine bleeding, follicular cysts, ovarian hyperstimulation, endometriosis, neoplasms, preeclampsia, placental insufficiency, respiratory distress, ascites, peritoneal sclerosis, adhesion formation, metastatic spreading, coronary artery disease, ischemic heart disease, ischemic limb disease, obesity, rheumatoid arthritis, synovitis, bone destruction, cartilage destruction, osteomyelitis, pannus growth, osterphyte formation, cancer, aseptic necrosis, impaired fracture healing, hepatitis, pneumonia, glomerulonephritis, asthma, nasal polyps, liver regeneration, pulmonary hypertension, systemic hypertension, diabetes, retinopathy of prematurity, diabetic retinopathy, choroidal disorders, intraocular disorders (e.g. age related macular degeneration), leukomafacia, stroke, vascular dementia, disease, thyroiditis, thyroid enlargement, thyroid pseudocyst, tumor metastasis, lymphoproliferative disorders, lympgoedema, AIDS, and hematologic malignancies.

13. A method of increasing the angiogenic process in a cell, a group of cells, or an organism, comprising administering a pharmaceutical composition which comprises a pharmaceutically effective amount of at least one gamma-secretase inhibitor or gamma-secretase pathway inhibitor to the cell, group of cells, or organism.

14. A method according to claim 13, wherein the pharmaceutical composition is administered to prevent, treat, or cure a condition selected from the group consisting of atherosclerosis, hemangioma, hemangioendothelioma, vascular malformations, warts, pyogenic granulomas, hair growth, Kaposi's sarcoma, scar keloids, allergic edema, neoplasms, psoriasis, decubitus or stasis ulcers, gastrointestinal ulcers, dysfunctional uterine bleeding, follicular cysts, ovarian hyperstimulation, endometriosis, neoplasms, preeclampsia, placental insufficiency, respiratory distress, ascites, peritoneal sclerosis, adhesion formation, metastatic spreading, coronary artery disease, ischemic heart disease, eschemic limb disease, obesity, rheumatoid arthritis, synovitis, bone destruction, cartilage destruction, osteomyelitis, pannus growth, osterphyte formation, cancer, aseptic necrosis, impaired fracture healing, hepatitis, pneumonia, glomerulonephritis, asthma, nasal polyps, liver regeneration, pulmonary hypertension, systemic hypertension, diabetes, retinopathy of prematurity, diabetic retinopathy, choroidal disorders, intraocular disorders (e.g. age related macular degeneration), leukomafacia, stroke, vascular dementia, disease, thyroiditis, thyroid enlargement, thyroid pseudocyst, tumor metastasis, lymphoproliferative disorders, lympgoedema, AIDS, and hematologic malignancies.

15. A method for screening for a substance which initiates or increases angiogenesis, comprising:
measuring an activity of a gamma-secretase pathway in the presence of a candidate compound in a suitable model;
measuring an activity of a gamma-secretase pathway in the absence of a candidate compound; and
comparing said activity in the presence of a candidate compound with said activity in the absence of the candidate compound,
wherein a change in activity indicates that said candidate initiates or increases angiogenesis.

16. An angiogenesis initiating or increasing medicament, comprising:
a pharmaceutically-effective amount of a gamma-secretase pathway inhibitor; and
a pharmaceutically-effective amount of at least one pro-angiogenic therapy.

17. An angiogenesis initiating or increasing medicament according to claim 16, wherein said gamma-secretase pathway inhibitor and said pro-angiogenic therapy are provided together as a single pharmaceutical composition.

18. An angiogenesis initiating or increasing medicament according to claim 16, wherein said gamma-secretase pathway inhibitor comprises an inhibitor selected from the group consisting of a dipeptide class gamma-secretase pathway inhibitor, a sulfonamide class gamma-secretase inhibitor, a transition state mimic class gamma-secretase inhibitor, a benzodiazepine class gamma-secretase inhibitor, and a benzocaprolactam class gamma-secretase inhibitor.

19. An angiogenesis initiating or increasing medicament according to claim 16, wherein said gamma-secretase pathway inhibitor is selected from the group consisting of DAPT, 1-(S)-endo-N-(1,3,3)-Trimethylbicyclo[2.2.1]hept-2-yl)-4-fluorophenyl Sulfonamide, WPE-III31C, S-3-[N′-(3,5-difluorophenyl-alpha-hydroxyacetyl)-L-alanilyl]amino-2,3-dihydro-1-methyl-5-phenyl-1H-1,4-benzodiazepin-2-one, and (N)-[(S)-2-hydroxy-3-methyl-butyryl]-1-(L-alaninyl)-(S)-1-amino-3-methyl-4,5,6,7-tetrahydro-2H-3-benzazepin-2-one.

20. An angiogenesis initiating or increasing medicament according to claim 16, wherein the pro-angiogenic therapy is selected from the group consisting of VEGF-A, VEGF-B, VEGF-C, VEGF-D, FGF-1, FGF-2, FGF-4, HIFalpha, and HGF.
